(19) United States
(12) Patent Application Publication
(10) Pub. No.: US 2025/0164391 A1
(43) Pub. Date: May 22, 2025

(54) Mechanical Property Inspection Device And Injection Molding System

(71) Applicant: SEIKO EPSON CORPORATION, Tokyo (JP)

(72) Inventor: Masayasu FUKUOKA, Shiojiri (JP)

(21) Appl. No.: 18/955,067

(22) Filed: Nov. 21, 2024

(30) Foreign Application Priority Data
Nov. 22, 2023 (JP) 2023-198593

(51) Int. Cl. G01N 21/3563; B29C 45/00; B29C 45/18; B29C 45/78; B29K 105/26; G01N 21/359; G01N 21/95; G01N 33/44

(52) U.S. Cl. CPC G01N 21/3563; B29C 45/0013; B29C 45/1866; B29C 45/78; G01N 21/359; G01N 21/95; G01N 33/442; B29C 2945/761; B29C 2945/76147; B29C 2945/76531; B29C 2945/76668; B29C 2945/76832; B29K 2105/26; G01N 2201/127

(57) ABSTRACT

A mechanical property inspection device is a mechanical property inspection device for inspecting the mechanical property of a molded article obtained by injection molding a resin material, and includes a spectral image acquisition section that acquires spectral images for a plurality of spectral wavelengths for the molded article; a spectral calculation section that calculates the spectral spectrum at a plurality of measurement points of the molded article from the spectral images for a plurality of the spectral wavelengths; a characteristic value calculation section that calculates a spectral characteristic value at a predetermined spectral wavelength in the spectral spectrum at a plurality of the measurement points; and a mechanical property estimation section that estimates the mechanical property of the molded article based on the spectral characteristic value.

The present application is based on, and claims priority from JP Application Serial Number 2023-198593, filed Nov. 22, 2023, the disclosure of which is hereby incorporated by reference herein in its entirety.

### BACKGROUND

### 1. Technical Field

The present disclosure relates to a mechanical property inspection device and an injection molding system provided with the mechanical property inspection device.

### 2. Related Art

A device for inspecting the characteristics of a resin molded article obtained by injection molding is known. In the device described in JP-A-2015-189211, light is irradiated from a first light irradiation section that is provided in a metal mold device or in a molding die clamping device onto a molded article after the mold is opened, and the light transmitted through the molded article is guided to a first light intensity detection section by a light guide member composed of an optical fiber or the like to be received. Molten molding material is irradiated with light from a second light irradiation section provided in a nozzle of an injection device, and the light transmitted through the molding material is received by a second light intensity detection section. Then, the degradation state of the molded article W is determined based on the light intensities detected by the first light intensity detection section and the second light intensity detection section.

However, in the apparatus described in JP-A-2015-189211, the light transmitted through one point of the molded article is guided to the first light intensity detection section by a light guiding member such as an optical fiber. In this case, the overall mechanical property of the molded article cannot be inspected. For example, there is a case where unevenness in mechanical strength occurs, such as degradation occurring only in a portion of a molded article. In this case, unless the degradation state of the entire molded article is detected, it is impossible to correctly determine whether the mechanical specification of the molded article is good or not.

Further, in JP-A-2015-189211 the intensity of light transmitted through the molding material is detected by the second light intensity detection section, but since the overall degradation state of the molded article after injection molding is not inspected, partial degradation cannot be detected in the same way as described above, and the mechanical property of the molded article cannot be correctly determined.

### SUMMARY

A mechanical property inspection device according to a first aspect of the present disclosure is a mechanical property inspection device for inspecting a mechanical property of a molded article obtained by injection molding a resin material, the mechanical property inspection device including a spectral image acquisition section that acquires spectral images for a plurality of spectral wavelengths for the molded article; a spectral calculation section that calculates the spectral spectrum at a plurality of measurement points of the molded article from the spectral images for a plurality of the spectral wavelengths; a characteristic value calculation section that calculates a spectral characteristic value at a predetermined spectral wavelength in the spectral spectrum at a plurality of the measurement points; and a mechanical property estimation section that estimates the mechanical property of the molded article based on the spectral characteristic value.

An injection molding system according to a second aspect of the present disclosure includes the mechanical property inspection device according to the first aspect described above and an injection molding machine for forming the molded article by injection molding, wherein the injection molding machine adjusts injection molding conditions based on an estimation result of the mechanical property by the mechanical property inspection device.

### BRIEF DESCRIPTION OF DRAWINGS

FIG. 1 is a schematic view showing configuration of an injection molding system of the present embodiment.

FIG. 2 is a diagram showing spectral spectrum measurement results of a plurality of molded articles having different degrees of degradation.

FIG. 3 is a diagram showing a second derivative spectrum obtained by second differentiating the spectral spectrum shown in FIG. 2.

FIG. 4 is a diagram showing an example of a detection calibration curve in the present embodiment.

FIG. 5 is a diagram showing an example of a property distribution image.

FIG. 6 is a flow chart showing a method of inspecting a mechanical property by the mechanical property inspection device of the present embodiment.

FIG. 7 is a diagram showing an example of specifying an inspection range of a molded article.

FIG. 8 is a flow chart showing a method of adjusting injection molding conditions of the present embodiment.

FIG. 9 is a diagram showing an example of a first property calibration curve for obtaining a molding condition from an average mechanical property value.

FIG. 10 is a diagram showing an example of a second property calibration curve for obtaining the molding conditions from the property variation value.

### DESCRIPTION OF EMBODIMENTS

Hereinafter, an embodiment of the present disclosure will be described.

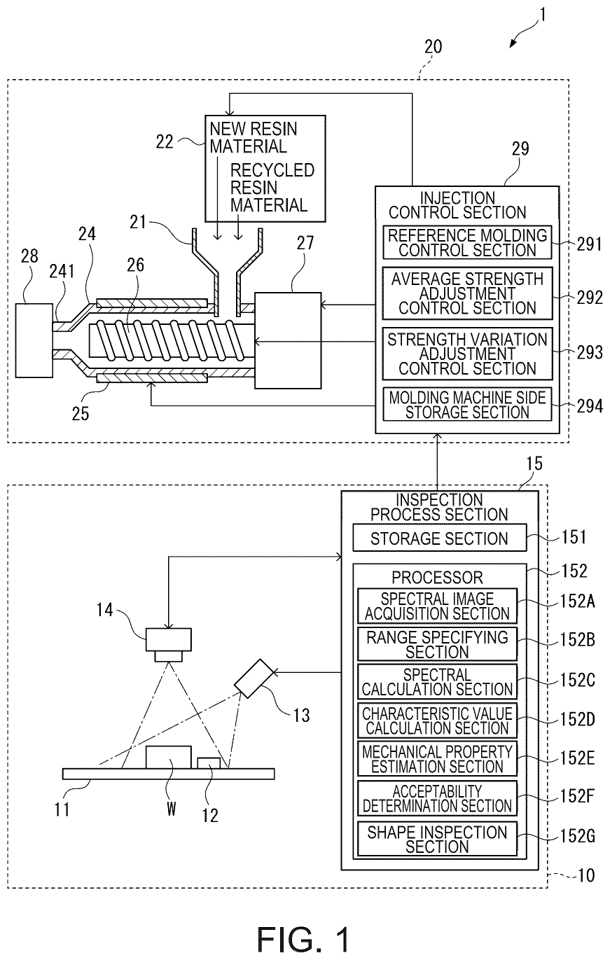

FIG. 1 is a schematic diagram showing configuration of an injection molding system 1 of the present embodiment.

In FIG. 1, the injection molding system 1 includes a mechanical property inspection device 10 for inspecting a mechanical property of a molded article W produced by injection molding, and an injection molding machine 20 for producing the molded article W by injection molding.

### Configuration of Mechanical Property Inspection Device 10

The mechanical property inspection device 10 is provided with a stage 11, a reference body 12, an illumination section 13, an imaging section 14, and an inspection process section 15.

The stage 11 is a placement base on which the molded article W, which is the target of inspection, is placed. The molded article W to be inspected may be all molded articles W produced by the injection molding machine 20, or may be molded articles W picked up at a predetermined cycle. In the present embodiment, a quick inspection can be performed by performing the mechanical property inspection based on a spectral image, and even when all the molded articles W are the target of inspection, a long time required for the mechanical property inspection can be avoided.

The reference body 12 has a reference spectrum. The reference spectrum is a reflectance spectrum of the reference body 12, and for example, light of each wavelength emitted from the illumination section 13 is reflected with a reflectance of 100% or approximately 100%. Note that the stage 11 may be coated with a reference color having no spectral unevenness, and in this case, the stage 11 can function as the reference body 12.

The illumination section 13 irradiates the molded article W and the reference body 12 with illumination light. As the illumination light, a broadband light source such as a halogen lamp is preferable. Note that if the spectrum (emission spectrum) of the illumination light is known, the gradation value of each pixel of the spectral image can be corrected using the emission spectrum.

The imaging section 14 is a spectroscopic camera for capturing a spectral image and, more specifically, is a hyperspectral camera capable of capturing a spectral image for a plurality of spectral wavelengths in a near-infrared region. The imaging section 14 captures a spectral image so that the whole of the molded article W placed on the stage 11 is contained in the image. The spectral image may be captured so that both the molded article W and the reference body 12 are included in the image, or the molded article W and the reference body 12 may be separately captured.

The inspection process section 15 can be a general computer such as a personal computer, a smartphone, or a tablet terminal. The inspection process section 15 controls the illumination section 13 and the imaging section 14, and performs an inspection process for the mechanical property of the molded article W based on a plurality of spectral images of the molded article W captured by the imaging section 14.

For example, the inspection process section 15 has a basic configuration of a general computer, such as a storage section 151 constituted by a memory or the like, and a processor 152 constituted by a CPU or the like. The processor 152 reads and executes programs recorded in the storage section 151, thereby functioning, as shown in FIG. 1, as a spectral image acquisition section 152A, a range specifying section 152B, a spectral calculation section 152C, a characteristic value calculation section 152D, a mechanical property estimation section 152E, an acceptability determination section 152F, and a shape inspection section 152G.

The spectral image acquisition section 152A controls the illumination section 13 and the imaging section 14, irradiates the molded article W and the reference body 12 with illumination light, captures spectral images of a plurality of spectral wavelengths of the molded article W and the reference body 12, and acquires (receives) the spectral images from the imaging section 14. Note that here an example is shown in which the spectral image acquisition section 152A controls the illumination section 13 and the imaging section 14 to capture spectral images, but the illumination section 13 and the imaging section 14 may be provided independently of the inspection process section 15. For example, a spectral image of the molded article W and the reference body 12 may be captured by another spectral image capturing apparatus and stored in a data server on the Internet, and the spectral image acquisition section 152A may download the spectral image from the data server to acquire the spectral image.

Various types of image processes such as a noise reduction process and pattern recognition may be applied to the acquired spectral image using various types of digital filters.

The range specifying section 152B specifies the inspection range of the molded article W from the spectral image. The inspection range may be specified by automatically detecting the contour of the molded article W from the spectral image by an edge detection filter and specifying the inside of the contour as the inspection range, or the inspection range in the spectral image may be specified by the user.

The spectral calculation section 152C uses a plurality of pixels included in the inspection range as measurement points and calculates the spectral spectrum of each measurement point. That is, the spectral calculation section 152C calculates the spectral spectrum of each pixel using each pixel value of the same pixel in the spectral images for a plurality of spectral wavelengths. Examples of the spectral spectrum include, but are not limited to, the spectral reflectance at each measurement point. In the present embodiment, the light reflected by the molded article W is captured by the imaging section 14, but when the molded article W has translucency, the light transmitted through the molded article W may be imaged and, in this case, the spectral calculation section 152C may calculate the transmittance spectrum as the spectral spectrum. The reflectance spectrum and the transmittance spectrum are calculated using the pixel values of the respective measurement points and the pixel values of the image captured from the reference body 12. The pixel value in the case of imaging the reference body 12 may be a pixel value of one point in the image of the reference body 12, or an average of pixel values of a plurality of points in the image of the reference body 12, in the spectral image for each spectral wavelength included in the reference body 12. In this example, the reflectance spectrum of each measurement point is calculated using the measured value of the reference body 12, but the pixel value of each measurement point may be used as it is as the spectral spectrum.

The characteristic value calculation section 152D calculates a spectral characteristic value at a predetermined characteristic wavelength at each measurement point based on the spectral spectrum. As the spectral characteristic value, for example in the present embodiment, a second derivative value of a spectral spectrum at an absorption peak wavelength corresponding to the resin material that is the material of the molded article W is used. Here, a relationship between the degradation state of the molded article W and the spectral characteristic value will be described.

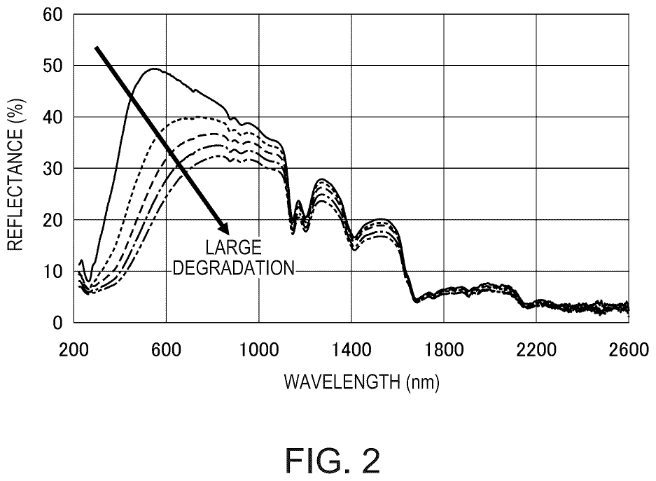

FIG. 2 is a diagram showing the spectral spectrum measurement results of a plurality of molded articles W having different degrees of degradation. As shown in FIG. 2, as degradation of the molded article W progresses, the spectral spectrum changes. That is, the functional group included in the resin structure of the molded article W changes depending on the degree of degradation, and the light absorption amount at the absorption peak wavelength originally in the molded article W (with no degradation) changes. Therefore, when reflectance with respect to a predetermined characteristic wavelength is compared in the spectral spectrum, the degradation trend of the resin material can be understood.

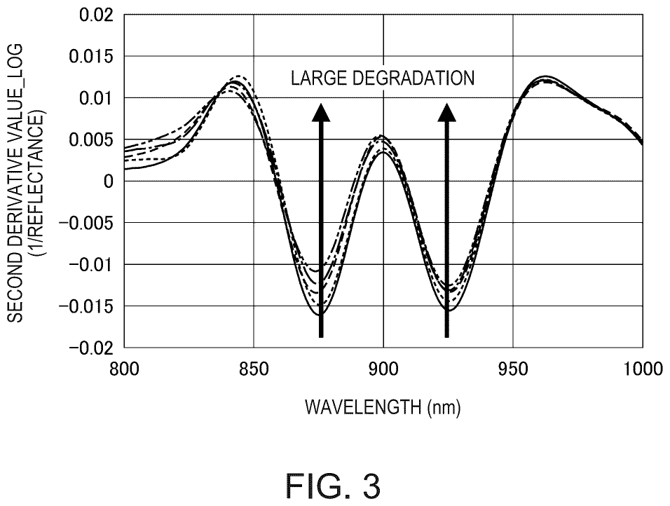

FIG. 3 is a diagram showing a second derivative spectrum obtained by second differentiating the spectral spectrum shown in FIG. 2. By second differentiating the spectral spectrum, the minimum value (i.e., absorption peak wavelength) in the spectral spectrum can be detected with high accuracy. When the absorption peak wavelength of the resin material is unknown, the absorption peak wavelength may be specified from the zero cross point of the first derivative spectrum.

The examples shown in FIGS. 2 and 3 are examples of the molded article W using recycled ABS resin, and it can be seen that absorption peak wavelengths appear at 875 nm and 925 nm, and the reflectance at 875 nm and 925 nm changes depending on the degree of degradation of the resin material. Therefore, the characteristic value calculation section 152D calculates the second derivative value at either or both of 875 nm and 925 nm as the spectral characteristic value.

The mechanical property estimation section 152E inspects the mechanical property of the molded article W based on the spectral characteristic value. As described above, there is a relationship between the degradation state of the molded article W and the spectral characteristic value, and the degradation trend of the molded article W can be estimated based on the spectral characteristic value.

More specifically, in the present embodiment, the property detection data (detection calibration curve) for property detection indicating the mechanical property of the molded article W with respect to the spectral characteristic value is stored in advance in the storage section 151, and the mechanical property value with respect to the spectral characteristic value calculated by the mechanical property estimation section is read. As the mechanical property value, for example, a known mechanical property such as Charpy impact strength can be raised as an example.

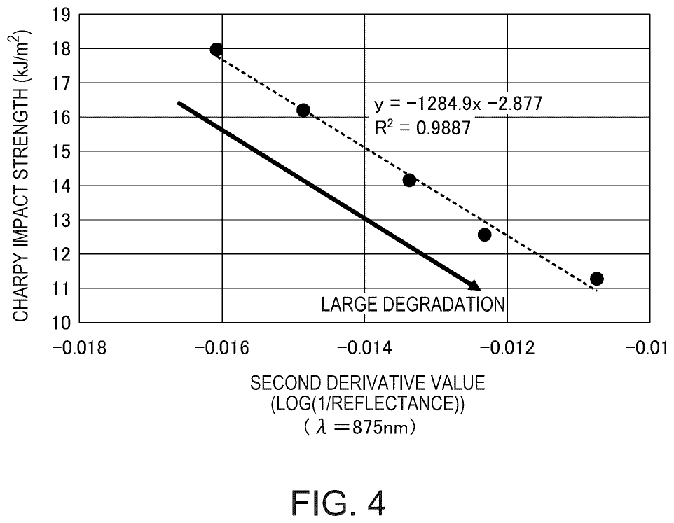

FIG. 4 is a diagram showing an example of the detection calibration curve.

As the detection calibration curve, for example, molded articles W in a plurality of degradation states are prepared, and the spectral characteristic value and the mechanical property value for each of the molded articles W are measured in advance. Also, the spectral characteristic value and the mechanical property value of each molded article W are plotted on a graph in which the horizontal axis is the spectral characteristic value (for example, the second derivative value) and the vertical axis is the mechanical property value (for example, the Charpy impact strength), and the first regression line is derived as a detection calibration curve. Note that although this example is an example in which the detection calibration curve is derived by a regression line, the detection calibration curve may be a curve equation or a higher-order function of two or more orders.

An example in which a detection calibration curve is used as the property detection data will be described, but it may be, for example, table data in which a plurality of spectral characteristic values and corresponding mechanical property values are recorded. In this case, the mechanical property estimation section 152E may read a mechanical property value that is closest to the calculated spectral characteristic value out from the characteristic detection data, or may calculate the mechanical property value by an interpolation method.

As described above, the mechanical property estimation section 152E calculates the mechanical property value for each measurement point, and then calculates the average mechanical property value of the whole of the molded article W and the property variation value indicating variation in the mechanical property.

For example, the mechanical property estimation section 152E calculates the arithmetic average of the mechanical property values of all the measurement points included in the inspection range as an average mechanical property value.

The mechanical property estimation section 152E calculates the property variation value based on the variance or standard deviation at the measurement points included in the inspection range.

The mechanical property estimation section 152E may further display a property distribution image in which the mechanical property values at the respective measurement points in the molded article W are superimposed on the molded article W on a display (not shown).

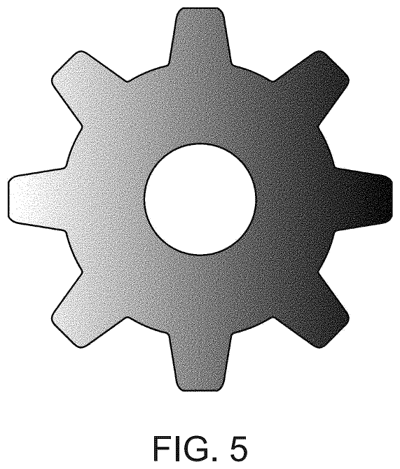

FIG. 5 is a diagram showing an example of the property distribution image. For example, a property distribution image obtained by coloring the outer shape image of the molded article W in colors corresponding to mechanical property values may be displayed, or a property distribution image obtained by changing the shade of the color according to the mechanical property value may be displayed. In the example of FIG. 5, the property distribution diagram is shown in the case where density is greater with greater degradation (lower mechanical strength). The mechanical property value may be binarized by a predetermined threshold value, and portions having a low mechanical property and portions having a large mechanical property may be separately displayed.

The acceptability determination section 152F performs acceptability determination of the molded article W based on the inspection result (estimation result) of the mechanical property. As the inspection determination of the mechanical property, a difference between the calculated average mechanical property value and a predetermined reference property value is calculated, and it is determined whether or not the difference is equal to or less than a predetermined first threshold value. When the difference between the average mechanical property value and the reference property value is equal to or smaller than the first threshold value, it is determined that the mechanical property is appropriate, and when the difference is larger than the first threshold value, it is determined that the mechanical property is abnormal (brittle).

The acceptability determination section 152F determines whether the calculated property variation value is equal to or greater than a predetermined second threshold value. When the property variation value is less than the second threshold value, it is determined to be normal, and when the property variation value is greater than the second threshold value, it is determined to be defective because a portion of the mechanical property is weak.

The shape inspection section 152G also functions as a foreign matter detection section of the present disclosure, and inspects for abnormalities in the shape of the molded article W and the presence or absence of foreign matter based on the spectral image.

The shape inspection section 152G specifies the contour of the molded article W, for example, by applying an edge detection filter or the like to the spectral image, and compares the external shape with external data of the molded article W stored in advance in the storage section 151, thereby inspecting for abnormalities in the outer shape of the molded article W and the presence or absence of foreign matter.

### Configuration of Injection Molding Machine 20

As shown in FIG. 1, the injection molding machine 20 includes a hopper 21, a resin introduction amount adjustment section 22, a cylinder 24, a heating section 25, a screw 26, an injection section 27, a molding die 28, and an injection control section 29.

The hopper 21 is an introduction port into which resin material, which will be the material of the molded article W, is introduced.

A resin introduction port is connected to the hopper 21. The resin introduction port is an introduction port into which the main material of the molded article W is introduced, and new recycled resin material, new resin material, and the like are introduced. Note that it is also possible to adopt a configuration in which the recycled resin material and the new resin material are introduced into the hopper 21 from separate introduction ports, and the introduction amounts of the recycled resin material and the new resin material can be adjusted.

The resin introduction amount adjustment section 22 adjusts the introduction ratio and the introduction amount of the recycled resin material and the new resin material of the resin material that will be the material of the molded article (W).

The cylinder 24 is a cylindrical member into which the resin material is introduced from the hopper 21. A nozzle 241 is provided at one end of the cylinder 24 and is connected to the molding die 28. An injection section 27 is connected to the other end of the cylinder 24.

The heating section 25 is a heater for heating and melting the resin material introduced into the cylinder 24. Note that the heating section 25 is provided in the cylinder 24 to heat and melt the resin material in the cylinder 24.

Note that, for example, a pre-plunger for heating and kneading may be provided in the passage from the hopper 21 to the cylinder 24. In this case, the pre-plunger is provided with the heating section 25 to heat and melt the resin material before it is introduced into the cylinder 24.

The screw 26 rotates about an axis to knead the resin material. The screw 26 is provided, for example, in the cylinder 24 to knead the resin material introduced into the cylinder 24. Note that as s described above, in the configuration in which the pre-plunger is provided in the path from the hopper 21 to the cylinder 24, the screw 26 may be provided in the pre-plunger to knead the resin material before it is introduced into the cylinder 24.

Note that hereinafter, a kneaded mixture obtained by heating and melting a resin material containing recycled resin material and new resin material and kneading the mixture using the screw 26 will be referred to as a molten resin material.

The injection section 27 applies pressure to the molten resin material in the cylinder 24 from the other end of the cylinder 24 and pushes the molten resin material out toward the nozzle 241. For example, as shown in FIG. 1, in a screw-in-line type injection molding machine 20, in which the screw 26 is provided in the cylinder 24, the injection section 27 moves the screw 26 back and forth in the axial direction in the cylinder 24. By this, the molten resin material pushed out by the screw 26 is injected from the nozzle 241 into the molding die 28.

Note that when the pre-plunger is provided in the path from the hopper 21 to the cylinder 24, the injection section 27 moves an injection ram inserted into the cylinder 24 forward and backward within the cylinder 24.

The molding die 28 is a die corresponding to the shape of the molded article W, and the molded article W is formed by introducing the molten resin injected from the cylinder 24.

Note that the hopper 21, the resin introduction amount adjustment section 22, the cylinder 24, the heating section 25, the screw 26, the injection section 27, and the molding die 28 may constitute one lot, and the injection molding machine 20 may comprise a plurality of lots.

The injection control section 29 controls the resin introduction amount adjustment section 22, the heating section 25, the screw 26, and the injection section 27. Specifically, the injection control section 29 includes a reference molding control section 291, an average strength adjustment control section 292, a strength variation adjustment control section 293, and a molding machine side storage section 294.

The reference molding control section 291 sets default molding conditions based on the reference conditions set in accordance with the molded article W to be molded. That is, the reference molding control section 291 sets an introduction amount of the resin material, an introduction ratio of new resin material to recycled resin material (hereinafter referred to as a resin introduction a ratio), heating temperature (plasticizing temperature) by the heating section 25, a screw rotation speed of the screw 26, and an injection pressure (for example, an injection speed and an injection amount) of the molten resin material to be injected by the injection section 27. Note that here, the ratio of the introduction amount of new resin material to the introduction amount of recycled resin material is referred to as the resin introduction ratio, but the ratio of the introduction amount of recycled resin material to the introduction amount of new resin material may be referred to as the resin introduction ratio.

The average strength adjustment control section 292 adjusts at least one of the resin introduction ratio and the plasticizing temperature according to the average mechanical property value output from the mechanical property inspection device 10.

In accordance with the property variation value output from the mechanical property inspection device 10, the strength variation adjustment control section 293 adjusts the plasticizing temperature, the screw rotation speed, the retention time that the molten resin material remains in the cylinder 24 (that is, the kneading time of the resin material and the master batch by the screw 26, and the injection interval by the injection section 27), and the injection pressure of the molten resin material.

### Mechanical Property Inspection Method for Injection Molding System 1

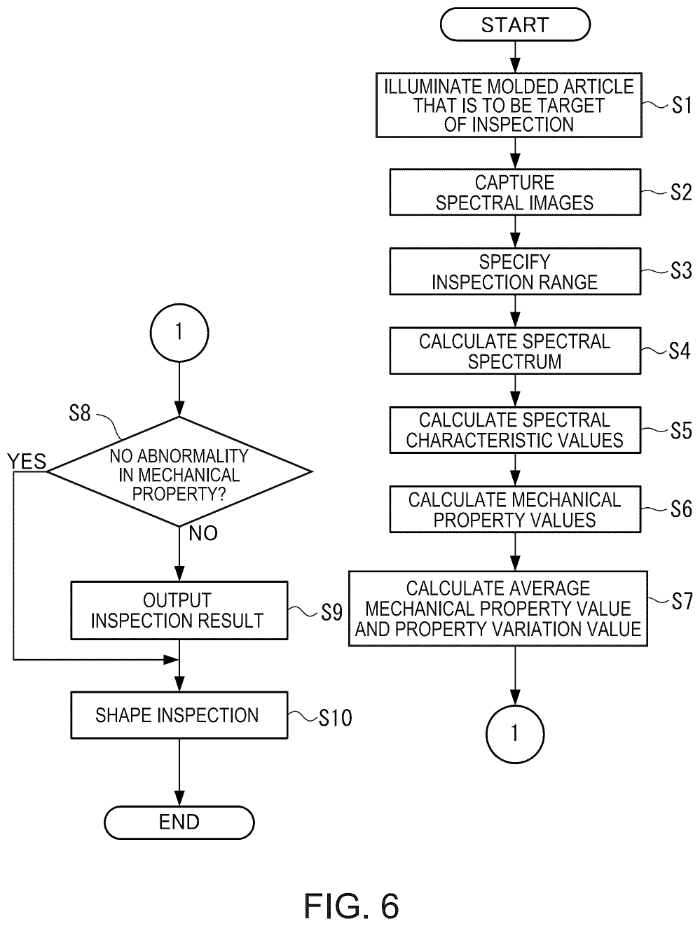

Next, a mechanical property inspection method in the injection molding system 1 will be described in more detail. FIG. 6 is a flow chart showing a mechanical property inspection method by the mechanical property inspection device 10. In the mechanical property inspection method by the mechanical property inspection device 10, a molded article W molded by the injection molding machine 20 of the injection molding system 1 is inspected as the target of inspection.

As for the molded article W to be the target of inspection, the molded article W may be randomly selected at an arbitrary timing as described above, or all molded articles W may be the target of inspection.

First, the molded article W that is to be the target of inspection is picked up and placed on the stage 11, the molded article W is illuminated by the illumination section 13 (Step S1), and then the spectral image acquisition section 152A controls the imaging section 14 to capture spectral images for a plurality of spectral wavelengths (Step S2). At the same time, a spectral image of the reference body 12 is also taken. In Step S2, an imaging process is performed with the relative position of the imaging section 14 and the molded article W fixed at the time of imaging each spectral image.

Next, the range specifying section 152B specifies the inspection range of the molded article W in each spectral image (Step S3).

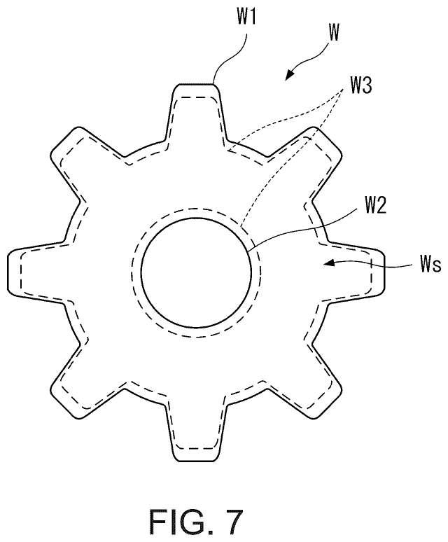

FIG. 7 is a diagram showing an example of specifying the inspection range of the molded article W. For example, the range specifying section 152B specifies the edge W1 (contour portion) of the molded article W using an edge detection process, and sets the inside of the edge W1 as an inspection range Ws. As shown in FIG. 7, when another closed edge W2 is detected inside the closed edge W1, the inspection range Ws may be specified as between the edge W1 and the edge W2. Note that since the luminance values at edges W1 and W2 greatly change, when the edges W1 and W2 are to be the targets of inspection, the accuracy of the mechanical property inspection is affected. Therefore, as shown in FIG. 7, the inspection range may be specified by connecting points (edge W3 in FIG. 7) separated from the edges W1 and W2 by a predetermined distance. Alternatively, the inspection range may be specified by the user.

Next, the spectral calculation section 152C calculates a spectral spectrum using each pixel in the inspection range Ws specified in Step S3 as a measurement point (Step S4).

For example, the spectral calculation section 152C performs the calculation by using pixel positions (x, y) of the spectral image as measurement points, and the reflectance R (x, y, λᵢ) of the spectral wavelength λᵢ is calculated at each measurement point (x, y) as R (x, y, λᵢ)=rᵢ(x, y)/r_{b}, based on the luminance value rᵢ(x, y) at the pixel positions (x, y) of the spectral image and the luminance value r_{b} with respect to the spectral wavelength λᵢ of the reference body 12. Note that here, i is a variable indicating the spectral wavelength, and is an integer from 1 to I. For example, when spectral images are captured at spectral wavelengths of 20 nm intervals in the visible light region of 400 nm to 700 nm, I=16. By this, the spectral spectrum (reflectance spectrum) as shown in FIG. 2 can be calculated for each measurement point.

Next, the characteristic value calculation section 152D calculates a spectral characteristic value from the calculated spectral spectrum of each measurement point (Step S5).

For example, the characteristic value calculation section 152D calculates the second derivative spectrum of the spectral spectrum at each measurement point, and calculates the second derivative value of a predetermined absorption peak wavelength as the spectral characteristic value.

Next, the mechanical property estimation section 152E calculates a mechanical property value (for example, Charpy impact strength) at each measurement point (Step S6) using the calculated spectral characteristic value and the detection calibration curve as shown in FIG. 4 that was stored in advance in the storage section 151.

The mechanical property estimation section 152E calculates an average mechanical property value and a property variation value of the whole of the molded article W from the mechanical property values calculated for the respective measurement points (Step S7).

At this time, the mechanical property estimation section 152E may display a property distribution image as shown in FIG. 5 on a display (not shown).

Thereafter, the acceptability determination section 152F determines whether or not there is an abnormality in the mechanical property of the molded article W (Step S8) based on the inspection result of the mechanical property.

For example, the acceptability determination section 152F calculates the difference (mechanical property evaluation value) between the reference mechanical strength set for each molded article W and the average mechanical property value calculated in Step S7, and determines that the molded article W is acceptable when the mechanical property evaluation value is less than a first threshold value, and determines that the molded article W is to be rejected when the mechanical property evaluation value is equal to or greater than the first threshold value.

Further, the acceptability determination section 152F calculates a difference (variation evaluation value) between the property variation value calculated in Step S7 and a reference variation value set in advance, and determines that it is acceptable when the variation evaluation value is less than a second threshold value, and determines that it is rejected when the variation evaluation value is equal to or greater than the second threshold value.

If a YES determination in Step S8, that is, if there is no abnormality in the mechanical property of the molded article W, then the inspection process of the mechanical property is terminated. In this case, it is determined that the molding conditions of the injection molding machine 20 are optimal, and the production of the molded article W by the injection molding machine 20 is continued while maintaining the current injection molding conditions.

On the other hand, when the determination in Step S8 is NO, that is, when there is an abnormality in the mechanical property of the molded article W, then the inspection result of the calculated mechanical property is output to the injection control section 29 of the injection molding machine 20 (Step S9). The inspection result may be, for example, an average mechanical property value and a property variation value, or may be a mechanical property evaluation value and a variation evaluation value, or both of these. By this, when Step S8 is determined to be NO, the injection control section 29 adjusts the molding conditions of the molded article W.

In the present embodiment, the shape inspection section 152G of the mechanical property inspection device 10 performs a shape inspection of the molded article W based on the spectral images acquired in Step S2 (Step S10). As an example of the timing of the shape inspection, the timing after the inspection of the mechanical property from Step S4 to Step S9 is indicated, but it may be after Step S2 or Step S3. Similarly to Step S3, for example, the shape inspection section 152G performs an edge detection process on the spectral image to detect the edges W1 and W2 of the molded article W, and compares the detected edges W1 and W2 with external shape data (reference data) of the molded article W that was previously recorded in the storage section 151. When the shapes of edges W1 and W2 are different from the external shape data, it is determined that the shape of the molded article W is abnormal, and when an edge different from the edges W1 and W2 is detected, it is determined that foreign matter is present.

The shape inspection result of the shape inspection section 152G may be appropriately displayed on the display.

### Adjustment of Molding Conditions of Injection Molding Machine 20

In the injection molding system 1 of the present embodiment, when the determination in Step S8 is NO, the molding conditions of the molded article W are adjusted, and control is performed so that the molded article W having the predetermined mechanical property is manufactured. When the injection molding system 1 is provided with a plurality of lots, the molded articles W molded in the respective lots are inspected by the mechanical property inspection device 10, and the molding conditions for the respective lots are individually adjusted based on the respective inspection results.

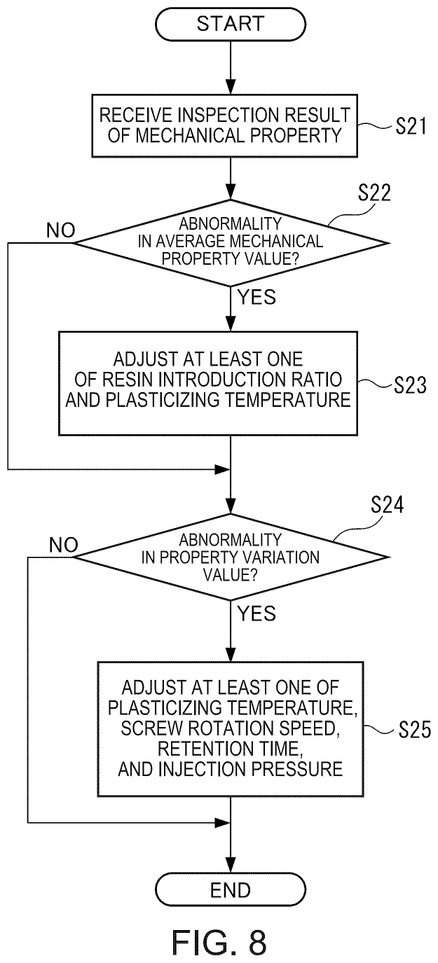

FIG. 8 is a flow chart showing a method of adjusting the injection molding conditions.

When the injection control section 29 receives the inspection result of the mechanical property from the mechanical property inspection device 10 (Step S21), the injection control section 29 determines whether or not the average mechanical property value (or the mechanical property evaluation value) is included in the received inspection result, that is, whether or not the average mechanical property value is abnormal (Step S22).

If YES is determined in Step S22, that is, if it is determined that there is an abnormality in the mechanical property of the molded article W as a whole, then the average strength adjustment control section 292 of the injection control section 29 controls at least one of the resin introduction amount adjustment section 22 and the heating section 25 to adjust at least one of the molding conditions of the resin introduction ratio and the plasticizing temperature in the resin material (Step S23). For example, the average strength adjustment control section 292 adjusts the resin introduction ratio while maintaining the plasticizing temperature constant. Alternatively, the average strength adjustment control section 292 adjusts the plasticizing temperature while maintaining the resin introduction ratio constant.

Here, as an example, a case where the plasticizing temperature is adjusted will be described.

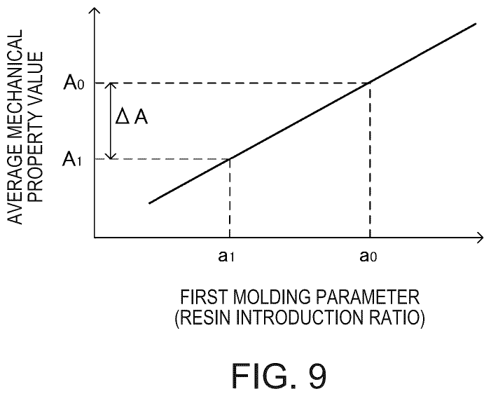

FIG. 9 shows an example of the first property calibration curve for obtaining the molding conditions from the average mechanical property value.

In the present embodiment, the first property calibration curve showing the relationship between the average mechanical property value and the molding condition as shown in FIG. 9 is stored in advance in the molding machine side storage section 294. Note that here, the molding conditions are referred to as “first molding parameters” in order to differentiate them from molding conditions for variations in mechanical property (to be described later).

The first molding parameter is a resin introduction ratio or a plasticizing temperature, and FIG. 9 shows an example in which a resin introduction ratio (an introduction ratio of new resin material to recycled resin material) is used as the first molding parameter.

Note that although the example of FIG. 9 shows the relationship of the mechanical property value to a single first molding parameter, the first property calibration curve may be shown on a three axis coordinate system in which the resin introduction ratio is on the X-axis, the plasticizing temperature is on the Z-axis, and the mechanical property value is on the Y-axis. That is, a function using the three variables of resin introduction ratio, plasticizing temperature, and mechanical property value may be used as the first property calibration curve.

In Step S23, using the first property calibration curve as described above, the average strength adjustment control section 292 changes the first molding parameter by a value corresponding to the difference AA between the average mechanical property value A₁ input from the mechanical property inspection device 10 and the target value (reference mechanical property value A₀) for the molded article W. For example, when the current resin introduction ratio is a₁ and the post-adjustment condition (the resin introduction ratio corresponding to the target value) is a₀, the resin introduction ratio is increased only by the amount a₀−a₁. By this, the first molding parameter is adjusted so that the difference (mechanical property evaluation value) between the average mechanical property value and the reference mechanical property value is less than the first threshold value.

Note that the above is an example of adjusting the resin introduction ratio, but the plasticizing temperature may be used as the first molding parameter and, in this case, a first property calibration curve showing the relationship between the average mechanical property value and the plasticizing temperature may be used.

After Step S23 and when the determination is NO in Step S22, the injection control section 29 determines whether or not the property variation value is included in the inspection result of the mechanical property received in Step S21 (Step S24).

If YES is determined in Step S24, that is, if it is determined that the molded article W has an abnormal variation in mechanical property, the strength variation adjustment control section 293 of the injection control section 29 adjusts at least one molding condition of the plasticizing temperature, the screw rotation speed, the retention time, and the injection pressure (Step S25). Here, the molding conditions are referred to as second molding parameters in order to distinguish them from the first molding parameters.

For example, the strength variation adjustment control section 293 may adjust one of the second molding parameters that determine the variation of the mechanical property and maintain the other parameters fixed, or it may adjust a plurality of second molding parameters.

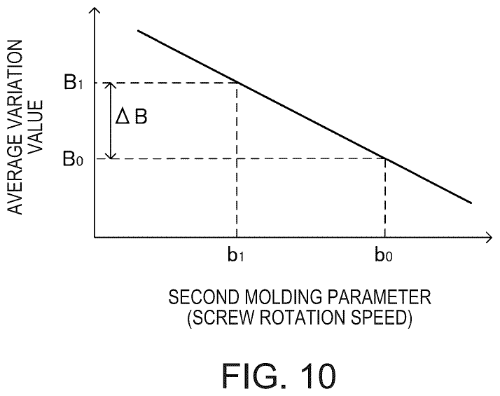

FIG. 10 is a diagram showing an example of the second property calibration curve for obtaining molding conditions from the property variation value.

In the second property calibration curve shown in FIG. 10, the second molding parameter is any one of the plasticizing temperature, the screw rotation speed, the retention time, and the injection pressure, for example, the screw rotation speed. Note that although the example of FIG. 10 shows the relationship of the property variation value with respect to a single second molding parameter, a function showing the relationship between a plurality of second molding parameters and the property variation value may be used as the second property calibration curve.

The adjustment of the second molding parameter is substantially the same as the adjustment of the average mechanical strength. That is, the second property calibration curve as shown in FIG. 10 is stored in advance in the molding machine side storage section 294. In Step S25, the strength variation adjustment control section 293 adjusts the second molding parameter based on the property variation value input from the mechanical property inspection device 10 and the second property calibration curve so that the difference (variation evaluation value) between the property variation value and the reference property variation value is less than the second threshold value. For example, the strength variation adjustment control section 293 changes the second molding parameter only by a value corresponding to a difference ΔB between the property variation value Bi input from the mechanical property inspection device 10 and a target value (reference variation value B₀) in the molded article W. For example, if the current screw rotation speed is b₁ and the post-adjustment condition (screw rotation speed corresponding to the target value) is b₀, then the screw rotation speed is decreased only by the amount of b₀−b₁. By this, the second molding parameter is adjusted so that the difference (variation evaluation value) between the property variation value and the reference variation value is less than the second threshold value. By this, the kneading performance by screw 26 is improved, and the variation in the mechanical property in the molded article W is improved.

Note that although the above is an example of adjusting the screw rotation speed, as described above, when the plasticizing temperature is to be adjusted, the strength variation adjustment control section 293 adjusts the heating temperature in the heating section 25. When the retention time is to be adjusted, the strength variation adjustment control section 293 adjusts the injection interval of the molten resin by the injection section 27. When the injection pressure is to be adjusted, the strength variation adjustment control section 293 adjusts the injection speed and the injection amount of the molten resin by the injection section 27.

### Effects of the Present Embodiment

An injection molding system 1 of the present embodiment is provided with a mechanical property inspection device 10 for inspecting the mechanical property of a molded article W obtained by injection molding a resin material. The processor 152 of the mechanical property inspection device 10 functions as the spectral image acquisition section 152A, the spectral calculation section 152C, the characteristic value calculation section 152D, and the mechanical property estimation section 152E. The spectral image acquisition section 152A acquires spectral images for a plurality of spectral wavelengths for the molded article W. The spectral calculation section 152C calculates spectral spectrum at a plurality of measurement points of the molded article W from the spectral images for the plurality of spectral wavelengths. The characteristic value calculation section 152D calculates spectral characteristic values at a predetermined spectral wavelength from the spectral spectrum of the plurality of measurement points. The mechanical property estimation section 152E estimates the mechanical property of the molded article W based on the spectral characteristic values.

In the present embodiment, the mechanical property of the molded article W is inspected based on the spectral images obtained by capturing the entire molded article W. Therefore, as compared with the case where one point of the molded article W is measured by a spot light, the average mechanical property of the entire molded article W and unevenness (variation) in the mechanical property can be appropriately measured. The structure can be simplified as compared with the case where the molded article W is scanned by changing the irradiation position of a spot light using a scanning mechanism or the case where the measurement position is changed by moving the relative position between a spot light and the molded article W using a moving mechanism.

In the mechanical property inspection device 10 of the present embodiment, the mechanical property estimation section 152E calculates a mechanical property value corresponding to a spectral characteristic value of each measurement point using a detection calibration curve indicating a relationship between mechanical property values indicating degradation states of the molded articles W molded from a resin material and spectral characteristic values. Also, the mechanical property estimation section 152E calculates the average value of the mechanical property values calculated for the respective measurement points as the average mechanical property value of the molded article W.

By using the pixels included in the inspection range as the measurement points, it is possible to calculate the mechanical property value of each position in a wide range of the molded article W. By calculating the arithmetic average of these mechanical property values, it is possible to obtain the overall average mechanical property value of the molded article W. In other words, when the mechanical property varies from place to place on the molded article W, the mechanical property of the molded article W cannot be correctly determined by the measuring a single point using a spot light. On the other hand, the arithmetic average of the mechanical property values at a plurality of points in the molded article W is a value indicating the trend of the mechanical property of the entire molded article W, so that the entire mechanical property of the molded article W can be properly inspected.

In the present embodiment, the mechanical property estimation section 152E calculates the mechanical property value corresponding to the spectral characteristic value of each measurement point using the detection calibration curve that indicates the relationship between the spectral characteristic value and the mechanical property value, which indicates the degradation state of the molded article W molded from the resin material. Further, the mechanical property estimation section 152E calculates variation (specific variation value) of the mechanical property value calculated for each measurement point.

As described above, by using the pixels included in the inspection range as the measurement points, it is possible to calculate the mechanical property value of each position in a wide range of the molded article W. The mechanical property estimation section 152E can determine the degree of in-plane variation of the mechanical property in the molded article W by calculating the property variation value. That is, when the property variation value is large, there is a point where the mechanical property differs greatly in the molded article W, that is, there is a position where the mechanical property is smaller than at other positions. In this case, a part of the molded article W will be easily damaged. In the present embodiment, it is possible to appropriately determine molded articles W that have such a large in-plane variation in mechanical property.

In the present embodiment, the spectral characteristic value is the second derivative value of the spectral spectrum at the absorption peak wavelength corresponding to the resin material of the molded article W.

The molded article W made of a resin has an absorption peak wavelength according to the resin material, and the spectral reflectance at the absorption peak wavelength changes according to the degradation state of the resin. Therefore, the degradation of the molded article W can be judged from the change of the reflectance at the absorption peak wavelength. The second derivative spectrum obtained by second differentiating the spectral spectrum has a maximum value or a minimum value even at the absorption peak wavelength. In the second derivative spectrum, the difference in the spectral shape other than the absorption peak wavelength is small, and the spectral shape at the absorption peak wavelength varies greatly according to the degradation of the resin. Therefore, when the second derivative spectrum is used, the absorption peak wavelength can be easily specified even when the absorption peak wavelength corresponding to the resin material is unknown or when the absorption peak wavelength changes according to the modification of the resin material, the introduction ratio of the recycled resin material and the new resin material contained in the resin material, or the introduction amount of the master batch or the like.

In the present embodiment, the processor 152 also functions as the acceptability determination section 152F, and determines whether or not the molded article W is a non-defective article based on the inspection result (estimation result) of the mechanical property value by the mechanical property estimation section 152E.

By this, it is possible to easily determine the average mechanical property of the molded article W and abnormality in variation of the mechanical property.

The injection molding system 1 of the present embodiment includes the above-described mechanical property inspection device 10 and the injection molding machine 20 for forming a molded article W by injection molding. Also, the injection molding machine 20 adjusts the injection molding conditions based on the inspection result (estimation result) of the mechanical property by the mechanical property inspection device 10.

By this, when variations in the average mechanical property or the mechanical property of the molded article W occur, the molding conditions can be adjusted in the injection molding machine 20 based on the inspection results of the mechanical property, and the defective product rate of the molded article W molded in the injection molding machine 20 can be reduced.

In the present embodiment, the injection molding machine 20 calculates a mechanical property value corresponding to the spectral characteristic value of each measurement point using a detection calibration curve that indicates the relationship between the spectral characteristic value and the mechanical property value, which indicates the degradation state of the molded article W, and calculates the average value of the mechanical property values calculated for each measurement point as the average mechanical property of the molded article W. The injection molding machine 20 is equipped with the hopper 21, the resin introduction amount adjustment section 22, the cylinder 24, and the heating section 25 and, based on the average mechanical property, at least one of the resin introduction ratio, which is the ratio of the introduction amount of the recycled resin material to the introduction amount of the new resin material, and the plasticizing temperature by heating section 25 is adjusted.

By this, by adjusting at least one of the resin introduction ratio and the plasticizing temperature based on the average mechanical property value, molded articles W having a proper mechanical property can be molded. In the case where it is difficult to adjust the average mechanical property value of the molded article W only by one of the resin introduction ratio or the plasticizing temperature, both the resin introduction ratio and the plasticizing temperature can be adjusted, and molded articles W having the desired average mechanical property can be molded.

In the present embodiment, the mechanical property estimation section 152E calculates a property variation value as the mechanical property variation of the molded article W. The injection molding machine 20 is provided with the hopper 21, the cylinder 24, the heating section 25, the screw 26, and the injection section 27, and at least one of the plasticizing temperature, the screw rotation speed, the retention time (kneading time), and the injection pressure is adjusted based on the mechanical property variation value.

By this, at least one of the plasticizing temperature, the screw rotation speed, the retention time, and the injection pressure can be adjusted based on the property variation value, and molded articles W having no variation in mechanical property can be molded. When it is difficult to adjust the in-plane unevenness in the mechanical property of the molded article W only by one of the plasticizing temperature, the screw rotation speed, the retention time, and the injection pressure, a plurality of or all of the plasticizing temperature, the screw rotation speed, the retention time, and the injection pressure can be adjusted, and molded articles W having no in-plane unevenness in the mechanical property can be molded.

In the present embodiment, the processor 152 of the mechanical property inspection device 10 also functions as the shape inspection section 152G and, based on the spectral image, inspects for foreign matter contained in the molded article W and shape abnormalities in the molded article W.

By this, it: is possible to determine not only abnormalities in the average mechanical property of the molded article W and variations in the mechanical property, but also abnormalities in the shape of the molded article W and foreign matter.

### Modifications

Note that the present disclosure is not limited to the above-described embodiments, and the present disclosure includes modifications, improvements, and configurations obtained by appropriately combining the embodiments within a range in which the object of the present disclosure can be achieved.

### First Modification

In the above embodiment, an example is shown in which the mechanical property inspection device 10 inspects both the average mechanical property and the in-plane unevenness of the mechanical property, but the mechanical property inspection device 10 may inspect only the average mechanical property or the in-plane unevenness of the mechanical property.

### Second Modification

The characteristic value calculation section 152D calculates, as the spectral characteristic value, the value (second derivative value) at the absorption peak wavelength of the second derivative spectrum obtained by second differentiating the spectral spectrum, but the present disclosure is not limited to this.

The characteristic value calculation section 152D may use the spectral reflectance at the absorption peak wavelength in the spectral spectrum as the spectral characteristic value.

Although the second derivative value and the reflectance at the absorption peak wavelength according to the resin material are exemplified as the spectral characteristic value, the second derivative value and the reflectance at other wavelengths may be used. In other words, as shown in FIG. 3, when the resin degrades, the reflectance also changes at wavelengths other than the absorption peak wavelength. Therefore, the reflectance or the first order differential value of any other wavelength may be used as the spectral characteristic value. For example, in a first differentiation spectrum obtained by f first differentiating a spectral spectrum, a first differentiation value of a wavelength taking a maximum value or a minimum value may be used as a spectral characteristic value.

As described above, when the absorption peak wavelength of the resin material is unknown, the absorption peak wavelength may be specified from the zero cross point of the first order differential spectrum. Alternatively, as described above, even when the absorption peak wavelength changes according to the modification of the resin material, the introduction ratio of the recycled resin material and the new resin material contained in the resin material, or the introduction amount of the master batch or the like, the absorption peak wavelength can be easily specified from the first order differential spectrum, and the second derivative value or the reflectance of the specified absorption peak wavelength can be calculated as the spectral characteristic value.

### Third Modification

Although the processor 152 of the mechanical property inspection device 10 functions as the shape inspection section 152G in the above embodiment, the injection control section 29 of the injection molding machine 20 may be configured to acquire spectral images from the mechanical property inspection device 10 and inspect the presence or absence of foreign matter or a shape abnormality.

### SUMMARY OF THE DISCLOSURE

A mechanical property inspection device according to a first aspect of the present disclosure is a mechanical property inspection device for inspecting a mechanical property of a molded article obtained by injection molding a resin material, the mechanical property inspection device including a spectral image acquisition section that acquires spectral images for a plurality of spectral wavelengths for the molded article; a spectral calculation section that calculates the spectral spectrum at a plurality of measurement points of the molded article from the spectral images for a plurality of the spectral wavelengths; a characteristic value calculation section that calculates a spectral characteristic value at a predetermined spectral wavelength in the spectral spectrum at a plurality of the measurement points; and a mechanical property estimation section that estimates the mechanical property of the molded article based on the spectral characteristic value.

With the mechanical property inspection device of this aspect, the mechanical property of the molded article is inspected based on spectral images obtained by imaging the entire molded article. Therefore, as compared with the case where a single point of the molded article is measured by a spot light, the average mechanical property of the entire molded article and the in-plane unevenness of the mechanical property can be appropriately measured. The structure can be simplified as compared with the case where the molded article is scanned by changing the irradiation position of the spot light using a scanning mechanism, or the case where the measured position is changed by moving the relative position between the spot light and the molded article by a moving mechanism.

In the mechanical property inspection device of this aspect, it is desirable that using a calibration curve indicating a relationship between the spectral characteristic value and a mechanical property value indicating a degradation state of the molded article that was molded from the resin material, the mechanical property estimation section calculates a mechanical property value corresponding to the spectral characteristic value at each of the measurement points and calculates an average value of the mechanical property values calculated for each of the measurement points as an average mechanical property of the molded article.

By this, by using the pixels included in the inspection range as the measurement points, it is possible to easily calculate mechanical property values at the respective positions in a wide range of the molded article and, by calculating the average mechanical property value of the arithmetic average, it is possible to inspect the mechanical property of the molded article as a whole.

In the mechanical property inspection device of the present aspect, the mechanical property estimation section can calculate a mechanical property value corresponding to the spectral characteristic value at each of the measurement points using a calibration curve indicating a relationship between the spectral characteristic value and a mechanical property value indicating a degradation state of the molded article that was molded from the resin material. Also, variation of the mechanical property value calculated for each of the measurement points is calculated as an in-plane variation of the mechanical property of the molded article.

By this, as in the above aspect, the mechanical property estimation section can easily calculate the mechanical property value corresponding to the spectral characteristic value at each measurement point using the calibration curve indicating the relationship between the spectral characteristic value and the mechanical property value, which indicates the degradation state of the molded article that was molded from the resin material. Then, by calculating the variation in the mechanical property values calculated for each measurement point, it is possible to inspect the in-plane variation of the mechanical property existing in the molded article.

In the mechanical property inspection device of the present aspect, the spectral characteristic value is a second derivative value of the spectral spectrum at an absorption peak wavelength corresponding to the resin material of the molded article.

The second derivative spectrum takes a minimum value at the absorption peak wavelength, and the change in the second derivative value at the absorption peak wavelength increases in accordance with the resin degradation state even when the change in the second derivative value at other wavelengths is small. Therefore, by using the second derivative value as the spectral characteristic value, the degradation state of the resin material can be appropriately determined. When the absorption peak wavelength corresponding to the resin material is unknown, the absorption peak wavelength can be easily specified even when the absorption peak wavelength changes according to changes in properties of the resin material, the introduction ratio between the recycled resin material and the new resin material contained in the resin material, or the introduction amount of the master batch or the like.

The mechanical property inspection device of the present aspect further includes an acceptability determination section for judging whether or not the molded article is a non-defective article based on an estimation result of the mechanical property by the mechanical property estimation section.

By this, based on the inspection result of the mechanical property of the molded article, a determination of a non-defective article of the molded articles can be made.

An injection molding system according to a second aspect of the present disclosure, includes the mechanical property inspection device according to the first aspect and an injection molding machine for forming the molded article by injection molding, wherein the injection molding machine adjusts injection molding conditions based on an estimation result of the mechanical property by the mechanical property inspection device.

By this, the injection molding conditions can be adjusted based on the inspection result of the mechanical property by the mechanical property inspection device so that abnormalities in the mechanical property do not occur, and the quality of the molded articles molded by the injection molding machine can be improved.

In the injection molding system of this aspect, using a calibration curve indicating a relationship between the spectral characteristic value and a mechanical property value indicating a degradation state of the molded article that was molded from the resin material, the mechanical property estimation section calculates a mechanical property value corresponding to the spectral characteristic value at each of the measurement points and calculates an average value of the mechanical property values calculated for each of the measurement points as an average mechanical property of the molded article and the injection molding machine is provided with a hopper into which resin material is introduced, a resin introduction amount adjustment section for adjusting an introduction ratio of recycled resin material and new resin material contained in the resin material introduced into the hopper, a cylinder into which the resin material introduced from the hopper is fed, and a heating section for heating and plasticizing the resin material in the cylinder, wherein at least one of the introduction ratio and a plasticizing temperature by the heating section is adjusted based on the average mechanical property.

By this, at least one of the introduction ratio of the recycled resin material to the new resin material and the plasticizing temperature of the resin material can be appropriately adjusted based on the average mechanical property value, and a molded article having a desired average mechanical property value can be molded.

In the injection molding system of this aspect, using a calibration curve indicating a relationship between the spectral characteristic value and a mechanical property value indicating a degradation state of the molded article that was molded from the resin material, the mechanical property estimation section calculates a mechanical property value corresponding to the spectral characteristic value at each of the measurement points and calculates variation of the mechanical property value calculated for each of the measurement points as a mechanical property variation of the molded article and the injection molding machine includes a hopper that introduces the resin material, a cylinder into which the resin material introduced from the hopper is fed, a heating section for heating and plasticizing the resin material in the cylinder, a screw that is inserted into the cylinder for kneading the resin material and that has adjustable rotation speed, and an injection section for pushing out the resin material in the cylinder, wherein at least one of plasticizing temperature, rotation speed of the screw, kneading time of the resin material by the screw, and injection pressure in the injection section is adjusted based on variation in the mechanical property.

By this, at least one of the plasticizing temperature, the screw rotation speed, the retention time, or the injection pressure can be adjusted based on the property variation value, and a molded article having no in-plane unevenness of the mechanical property can be molded.

In this aspect of the injection molding system, it is desirable to further include a foreign matter detection section configured to detect foreign matter contained in the molded article based on the spectral image.

By this, it is possible to further inspect whether or not the molded article formed by the injection molding system contains foreign matter.

In this aspect of the injection molding system, it is desirable to further include a shape inspection section configured to inspect a shape of the molded article based on the spectral image.

By this, further inspection of molded articles formed in the injection molding system for shape anomalies can be done.

What is claimed is:

1. A mechanical property inspection device for inspecting a mechanical property of a molded article obtained by injection molding a resin material, the mechanical property inspection device comprising:
a spectral image acquisition section that acquires spectral images for a plurality of spectral wavelengths for the molded article;
a spectral calculation section that calculates the spectral spectrum at a plurality of measurement points of the molded article from the spectral images for a plurality of the spectral wavelengths;
a characteristic value calculation section that calculates a spectral characteristic value at a predetermined spectral wavelength in the spectral spectrum at a plurality of the measurement points; and
a mechanical property estimation section that estimates the mechanical property of the molded article based on the spectral characteristic value.

2. The mechanical property inspection device according to claim 1, wherein
using a calibration curve indicating a relationship between the spectral characteristic value and a mechanical property value indicating a degradation state of the molded article that was molded from the resin material, the mechanical property estimation section calculates a mechanical property value corresponding to the spectral characteristic value at each of the measurement points and calculates an average value of the mechanical property values calculated for each of the measurement points as an average mechanical property of the molded article.

3. The mechanical property inspection device according to claim 1, wherein
using a calibration curve indicating a relationship between the spectral characteristic value and a mechanical property value indicating a degradation state of the molded article that was molded from the resin material, the mechanical property estimation section calculates a mechanical property value corresponding to the spectral characteristic value at each of the measurement points and calculates variation of the mechanical property value calculated for each of the measurement points as an in-plane variation of the mechanical property of the molded article.

4. The mechanical property inspection device according to claim 1, wherein
the spectral characteristic value is a second derivative value of the spectral spectrum at an absorption peak wavelength corresponding to the resin material of the molded article.

5. The mechanical property inspection device according to claim 1, further comprising:
an acceptability determination section for judging whether or not the molded article is a non-defective article based on an estimation result of the mechanical property by the mechanical property estimation section.

6. An injection molding system comprising:
the mechanical property inspection device according to claim 1 and
an injection molding machine for forming the molded article by injection molding, wherein
the injection molding machine adjusts injection molding conditions based on an estimation result of the mechanical property by the mechanical property inspection device.

7. The injection molding system according to claim 6, wherein
using a calibration curve indicating a relationship between the spectral characteristic value and a mechanical property value indicating a degradation state of the molded article that was molded from the resin material, the mechanical property estimation section calculates a mechanical property value corresponding to the spectral characteristic value at each of the measurement points and calculates an average value of the mechanical property values calculated for each of the measurement points as an average mechanical property of the molded article and
the injection molding machine is provided with a hopper into which resin material and filler are introduced, a resin introduction amount adjustment section for adjusting an introduction ratio of recycled resin material and new resin material contained in the resin material introduced into the hopper, a cylinder into which the resin material introduced from the hopper is fed, and a heating section for heating and plasticizing the resin material in the cylinder, wherein at least one of the introduction ratio and a plasticizing temperature by the heating section is adjusted based on the average mechanical property.

8. The injection molding system according to claim 6, wherein
using a calibration curve indicating a relationship between the spectral characteristic value and a mechanical property value indicating a degradation state of the molded article that was molded from the resin material, the mechanical property estimation section calculates a mechanical property value corresponding to the spectral characteristic value at each of the measurement points and calculates variation of the mechanical property value calculated for each of the measurement points as a mechanical property variation of the molded article and
the injection molding machine includes a hopper that introduces the resin material, a cylinder into which the resin material introduced from the hopper is fed, a heating section for heating and plasticizing the resin material in the cylinder, a screw that is inserted into the cylinder for kneading the resin material and that has adjustable rotation speed, and an injection section for pushing out the resin material in the cylinder, wherein at least one of plasticizing temperature, rotation speed of the screw, kneading time of the resin material by the screw, and injection pressure in the injection section is adjusted based on variation in the mechanical property.

9. The injection molding system according to claim 6, further comprising:
a foreign matter detection section configured to detect foreign matter contained in the molded article based on the spectral image.

10. The injection molding system according to claim 6, further comprising:
a shape inspection section configured to inspect a shape of the molded article based on the spectral image.
